(19) United States
(12) Patent Application Publication
(10) Pub. No.: US 2026/0131371 A1
(43) Pub. Date: May 14, 2026

(54) METHOD AND COMPOSITE PREFORM FOR PRODUCING A SHAPED METAL PRODUCT

(71) Applicant: Heraeus Medevio GmbH & Co. KG, Hanau (DE)

(72) Inventors: Bernd SPANIOL, Hanau (DE); Tim ESPOSTO, Hanau (DE); Andre WEIGAND, Hanau (DE); Markus JUNG, Hanau (DE); Claudia DRESCHER, Hanau (DE)

(21) Appl. No.: 19/381,519

(22) Filed: Nov. 6, 2025

(30) Foreign Application Priority Data
Nov. 12, 2024 (DE) 102024132993.9

(51) Int. Cl. B21C 23/04; A61N 1/05; C21D 1/26; C22F 1/10; C22F 1/14

(52) U.S. Cl. CPC B21C 23/04; C21D 1/26; C22F 1/10; C22F 1/14; A61N 1/05

(57) ABSTRACT

A method for producing a shaped metal product, wherein a metal preform containing an inner sacrificial element in a cavity is shaped to smaller dimensions and the sacrificial element is subsequently removed. The invention further comprises a composite material for producing a shaped metal product and its use. The metal products can be used to produce an implantable medical device.

### CROSS-REFERENCE TO RELATED APPLICATIONS

This application claims priority pursuant to 35 U.S.C. 119(a) to German Application No. 102024132993.9, filed Nov. 12, 2024, which application is incorporated herein by reference in its entirety.

### FIELD

The present invention relates to a method for producing a shaped metal product, wherein a metal preform containing an inner sacrificial element in a cavity is shaped to smaller dimensions and the sacrificial element is subsequently removed. The invention further comprises a composite material for producing a shaped metal product and its use. The metal products can be used to produce an implantable medical device.

### BACKGROUND OF THE INVENTION

Electrodes or electrode parts for medical devices, preferably both active and passive implantable medical devices, typically have very small dimensions, with the trend in this technical field being towards further miniaturization.

Conventionally, medical electrodes or electrode parts require very expensive and complex production facilities and methods with many individual method steps. For example, ring electrodes for medical devices are often made from a bar material by machining such as turning, and the excess material inside the ring is removed by electrical discharge machining, for example. Such conventional production methods are costly and may have limitations regarding the shape of the electrode or part thereof and the achievable degree of miniaturization.

EP 3 950 046 A1 describes a method for producing a shaped metal product which comprises providing an outer sacrificial element, an inner sacrificial element, and a metal preform arranged between the outer and inner sacrificial elements. After a forming process and separation of a composite preform disk, the sacrificial elements are removed to obtain the shaped metal product. A disadvantage of the described method is that during the shaping process, which is advantageously a drawing process, geometric deformations occur, particularly in the metal preform, which change the geometry of the final shaped metal product in an uncontrolled manner. Due to the small dimensions of the shaped metal products, which are preferably used as electrodes or electrode components in medical devices, these geometric deformations result in high production waste, as the shaped metal products no longer meet the high requirements for low manufacturing tolerance.

There is therefore a market demand for an improved production method for such shaped metal products, for example for medical devices, with the smallest possible manufacturing tolerances. In particular, the present invention is directed to providing a method for producing a shaped metal product which offers high flexibility with respect to the dimensions, geometry and/or shaping of a ring electrode or a segmented electrode for a medical device. The method of the present invention can be used in particular for the production of electrodes or segmented electrodes for medical devices, in particular for electrical stimulation and/or in sensor technology.

### SUMMARY

One object of the present invention is to overcome, at least in part, one or more of the disadvantages resulting from the prior art.

It is a further object of the invention to provide a method for producing a shaped metal product which offers the highest possible flexibility with respect to the dimensions, geometry and/or shaping of the shaped metal products for a medical device and, at the same time, has the lowest possible manufacturing tolerances, i.e. a high process stability.

### PREFERRED EMBODIMENTS OF THE INVENTION

A contribution to the at least partial fulfillment of at least one of the aforementioned objects is made by the features of the independent claims. The dependent claims provide preferred embodiments that contribute to the at least partial fulfillment of at least one of the objects.

A first embodiment of the invention is a method for producing a shaped metal product, preferably for a medical device, in particular for producing an electrode or part of an electrode for a medical device, comprising the following method steps:
a) providing a metal preform having a cavity A;
b) providing at least one inner sacrificial element;
c) producing an assembled preform by inserting the inner sacrificial element into the cavity A of the metal preform;
d) processing the assembled preform produced in method step c) to obtain a form-fitting preform, wherein in the form-fitting preform the metal preform abuts the inner sacrificial element substantially in a form-fitting manner;
e) diffusion annealing of the form-fitting preform produced in method step d) to obtain a composite preform having an outer diameter D1;
f) shaping the composite preform obtained in method step e) to obtain a shaped composite preform having a smaller outer diameter D2 than the outer diameter D1 of the composite preform obtained in method step e);
g) separating a composite preform disk from the shaped composite preform obtained in method step f);
h) removing the inner sacrificial element from the composite preform disk obtained in method step g) to obtain the shaped metal product.

In a preferred embodiment of the method, in method step b) an outer sacrificial element having a cavity B is additionally provided, method step c) comprises producing an assembled preform by inserting the metal preform into the cavity B of the outer sacrificial element and inserting the inner sacrificial element into the cavity A of the metal preform, in method step d) the assembled preform is processed such that, in the form-fitting preform, the metal preform abuts the inner sacrificial element and the outer sacrificial element substantially in a form-fitting manner, and in process step h), the outer sacrificial element is additionally removed. This embodiment is a second embodiment of the invention, which is preferably dependent upon the first embodiment of the invention.

In a preferred embodiment of the method, the processing in method step d) comprises a drawing process, wherein the drawing process is preferably carried out with a deformation factor between 5% and 40% and/or using a drawing die with an inclusion angle 2 a in a range of 5° to 20°, or the processing in method step d) comprises a pressing process, in particular an isostatic pressing. This embodiment is a third embodiment of the invention, which is preferably dependent upon the first or second embodiment of the invention.

In a preferred embodiment of the method, the diffusion annealing in method step e) is carried out in a temperature range of 450° C. to 1200° C., preferably in a temperature range of 450° C. to 1150° C. This embodiment is a fourth embodiment of the invention, which is preferably dependent upon one of the preceding embodiments of the invention.

In a preferred embodiment of the method, the diffusion annealing in method step e) is carried out over a period of time in a range from 15 min to 300 min, preferably in a range from 20 min to 260 min. This embodiment is a fifth embodiment of the invention, which preferably depends upon one of the preceding embodiments of the invention.

In a preferred embodiment of the method, the shaping in method step f) comprises a drawing process, wherein the drawing process is preferably carried out with a deformation factor between 5% and 40% and/or using a drawing die with an inclusion angle 2 a in a range of 5° to 20°. This embodiment is a sixth embodiment of the invention, which preferably depends upon one of the preceding embodiments of the invention.

In a preferred embodiment of the method, the drawing process in method step f) comprises 3 to 50 individual drawing steps. This embodiment is a seventh embodiment of the invention, which is preferably dependent upon the sixth embodiment of the invention.

In a preferred embodiment of the method, the composite preform is annealed in method step f) between the individual drawing steps. This embodiment is an eighth embodiment of the invention, which is preferably dependent upon the sixth or seventh embodiment of the invention.

In a preferred embodiment of the method, the metal preform comprises a metal selected from the group consisting of platinum, iridium, steel, titanium, hafnium, niobium, cobalt, nickel, chromium, zirconium, rhenium, tungsten, molybdenum, and alloys of at least one of these metals, preferably selected from the group consisting of nickel-cobalt alloys, steel, platinum and platinum-iridium alloys. This embodiment is a ninth embodiment of the invention, which is preferably dependent upon one of the preceding embodiments of the invention.

In a preferred embodiment of the method, the composite preform is shaped so that it assumes a smaller dimension without substantially altering its overall geometry. This embodiment is a tenth embodiment of the invention, which preferably depends upon one of the preceding embodiments of the invention.

An eleventh embodiment of the invention is a composite preform for producing a shaped metal product, comprising
a) a metal preform having a cavity A′,
b) an inner sacrificial element arranged in the cavity A′ of the metal preform,
characterized in that an inner diffusion zone with a thickness in a range of 1 μm to 15 μm is formed at an interface between the metal preform and the inner sacrificial element.

In a preferred embodiment of the composite preform, the inner diffusion zone comprises an intermetallic phase. This embodiment is a twelfth embodiment of the invention, which is preferably dependent upon the eleventh embodiment of the invention.

In a preferred embodiment of the composite preform comprising an outer sacrificial element having a cavity B′, wherein the metal preform is arranged in the cavity B′ of the outer sacrificial element and an outer diffusion zone with a thickness in a range of 1 μm to 15 μm is formed at an interface of the metal preform and the outer sacrificial element. This embodiment is a thirteenth embodiment of the invention, which is preferably dependent on the eleventh or twelfth embodiment of the invention.

In a preferred embodiment of the composite preform, the outer diffusion zone comprises an intermetallic phase. This embodiment is a fourteenth embodiment of the invention, which is preferably dependent upon the thirteenth embodiment of the invention.

A fifteenth embodiment of the invention relates to a use of a composite preform according to any of the eleventh to fourteenth embodiments of the invention for producing a medical product, preferably for producing a medical ring electrode or a segmented medical electrode.

### General

With respect to the embodiments described herein, the elements of which “have,” or “comprise,” a particular feature (for example, a material), in principle, a further embodiment is always contemplated in which the relevant element consists solely of the feature, i.e., does not comprise any other constituents. The word “comprise” or “comprising” is used herein synonymously with the word “have” or “having.”

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, it is contemplated in principle that all features described herein in connection with a method are also applicable to the products and devices described herein, and vice versa.

All such considered combinations are not explicitly listed in all instances, simply in order to keep the description brief. Technical solutions known to be equivalent to the features described herein are also intended in principle to be encompassed by the scope of the invention.

In the present description, specifications of ranges also contain the values specified as limits. A specification of the type “in the range from X to Y” with respect to a quantity A consequently means that A can take the values X, Y and values between X and Y. Ranges which are limited on one side, of the type “up to Y” for a size A, accordingly mean a value Y and less than Y.

Some of the features described are associated with the term “substantially.” The term “substantially” is to be understood as meaning that, under real conditions and production techniques, a mathematically exact interpretation of terms such as “overlap” “perpendicular,” “diameter” or “parallelism” can never be given exactly, but only within certain manufacturing tolerances. For example, “substantially perpendicular axes” enclose an angle of 85 degrees to 95 degrees relative to one another, and “substantially equal volumes” comprise a variation of up to 5% by volume. For example, a “device consisting substantially of plastics” comprises a plastics content of ≥95 to ≤100% by weight. For example, a “substantially complete filling of a volume B” comprises a filling of ≥95 to ≤100% by volume of the total volume of B.

### DETAILED SUMMARY

A first object of the invention relates to a method for producing a shaped metal product, preferably for a medical device, comprising the following method steps:
a) providing a metal preform having a cavity A;
b) providing an inner sacrificial element;
c) producing an assembled preform by inserting the inner sacrificial element into the cavity A of the metal preform;
d) processing the assembled preform produced in method step c) to obtain a form-fitting preform, wherein in the form-fitting preform the metal preform abuts the inner sacrificial element substantially in a form-fitting manner;
e) diffusion annealing of the form-fitting preform produced in method step d) to obtain a composite preform having an outer diameter D1;
f) shaping the composite preform obtained in method step e) to obtain a shaped composite preform having a smaller outer diameter D2 than the outer diameter D1 of the composite preform obtained in method step e);
g) separating a composite preform disk from the shaped composite preform obtained in method step f);
h) removing the inner sacrificial element from the composite preform disk obtained in method step g) to obtain the shaped metal product.

The method is used to produce a shaped metal product which can preferably be designed to be flexible in its dimensions, geometry and/or shape. The method makes it possible to uniformly deform a, preferably monolithic, metal preform, in particular in the longitudinal direction, in order to produce a shaped metal product which has substantially the same geometry and/or shape as the metal preform, but has a reduced diameter. This is achieved by forming a composite preform by means of diffusion annealing, which composite preform comprises the metal preform and at least one inner sacrificial element and which can be stretched with low manufacturing tolerance while maintaining its geometry and/or shape to obtain a reduced diameter.

It is understood that the desired final geometry and shape of the shaped metal product can be fully produced at the level of the upscaled metal preform. Thus, the method according to the invention enables access to miniaturized metal products with any geometry and/or shape. Using conventional methods, such metal products would only be accessible, if at all, through very complex and cost-intensive processes.

In method step (a), a metal preform is provided. The metal preform comprises at least one cavity A. The cavity may have any shape when viewed in a radial cross section. For example, the cavity may have a circular cross section. Furthermore, the cavity A, when viewed in a radial cross section, may have a C-shaped, U-shaped, or V-shaped cross section, for example. This refers to a shape that resembles the character mentioned above.

In principle, the metal preform can have any possible shape. Preferably, the metal preform has a homogeneous shape along a longitudinal axis of the metal preform, i.e. it has a fixed cross-sectional profile.

The metal preform can have a closed shape, such as a hollow cylindrical shape with a circular cross section, or an open shape. In one embodiment, the outer shape of the metal preform, when viewed in a radial cross section, has an open or a closed outer shape. An “open outer shape” means that the metal preform comprises a cavity A that is not completely surrounded by the material of the metal preform in the radial cross section. For example, a metal preform with a C-shaped radial cross section is considered an open outer shape, while a metal preform with a completely circular or O-shaped radial cross section is considered a closed outer shape. A closed shape of the metal preform can have a cross-sectional shape selected from the list of oval, polygonal, cubic, rectangular and round. The metal preform can, for example, consist of a tube comprising the cavity A. Such a tube can, for example, have an oval, polygonal, cubic, rectangular or circular radial cross-sectional shape.

In a further embodiment, the metal preform has an open outer shape, such as a U-shape, L-shape, T-shape, S-shape or H-shape. This refers to a shape that resembles the characters mentioned above.

In a further variant of the method, the inner shape of the metal preform is not circular, as can be seen in a radial cross-section. The “inner shape” of the monolithic metal preform refers to the cross-sectional surfaces of the metal preform that do not face outwards. For example, the inner shape comprises an open recess, depression, indentation or an open or closed cavity.

The metal preform can be provided in different ways. For example, the metal preform can be produced and provided using one or more methods selected from the group consisting of electrical discharge machining (EDM), additive manufacturing processes such as laser melting or selective electron beam melting, deep hole drilling, turning and milling.

The material of the metal preform can be any metal suitable for producing the desired shaped metal product. Preferably, the metal of the metal preform is a biocompatible metal. The term “biocompatible” within the meaning of the present invention refers to a material that is considered by a person skilled in the art to be safe to come into contact with a living organism, such as a human, for a certain period of time, for example when used in an implantable medical device. The person skilled in the art knows or can determine whether a metal is biocompatible or not. The biocompatible metal can, for example, be a biocompatible metal in accordance with EN ISO 10993.

Preferably, the metal preform comprises a metal selected from the group consisting of platinum, gold, iridium, steel, titanium, hafnium, niobium, tantalum, cobalt, nickel, chromium, zirconium, rhenium, tungsten, molybdenum and alloys of at least one of these metals. Preferably, the metal is selected from the group consisting of nickel-cobalt alloys, steel, platinum, platinum-iridium alloys and nitinol. Preferably, the steel is stainless steel. Preferably, the nickel-cobalt alloy is MP35N. Preferably, the platinum-iridium alloy is PtIr10 or PtIr20, particularly preferably PtIr10.

MP35N is a nickel-cobalt alloy containing nickel, cobalt, chromium and molybdenum. A variant of MP35 is described in the industry standard ASTM F562-13. In one embodiment, MP35 is an alloy containing 33% to 37% Co, 19% to 21% Cr, 9% to 11% Mo, and 33% to 37% Ni.

PtIr10 is an alloy consisting of 88% to 92% platinum and 8% to 12% iridium.

PtIr20 is an alloy consisting of 78% to 82% platinum and 18% to 22% iridium.

Nitinol is a nickel-titanium shape memory alloy with an ordered cubic crystal structure and a nickel content of approximately 45-60%, with the remaining content being titanium. Nitinol has good properties in terms of biocompatibility and corrosion resistance.

Unless otherwise stated, all percentages contained herein are to be understood as mass percentages (percentage by weight).

In method step b), an inner sacrificial element is provided. The inner sacrificial element preferably has an outer shape which substantially corresponds to the inner shape, i.e. the cavity A, of the metal preform.

The material of the inner sacrificial element can be chosen arbitrarily as long as it is selectively removable from the material of the metal preform in the composite preform disk. The material of the inner sacrificial element must therefore be selected depending on the material of the metal preform.

In one embodiment of the method, the inner sacrificial element comprises a base metal or a base metal alloy, preferably being made of a base metal or a base metal alloy. A “base” metal or a “base” metal alloy within the meaning of the present invention is a metal that is not selected from the group consisting of ruthenium, rhodium, palladium, silver, osmium, iridium, platinum, gold, mercury and alloys of the aforementioned metals.

The base metal or base metal alloy is preferably selected from the group consisting of zinc, tin, nickel, copper, steel and alloys of any of these metals. The base metal or base metal alloy may react with acids, preferably strong acids (e.g. sulfuric acid, hydrochloric acid, nitric acid).

In a preferred embodiment of the method, the inner sacrificial element comprises a metal selected from the group consisting of nickel, copper, steel and alloys of any of these metals; preferably, the inner sacrificial element is made of one of these metals or one of these alloys.

In method step c) of the method, an assembled preform is produced by inserting the inner sacrificial element into the cavity A of the metal preform. For example, the metal preform has a hollow cylindrical shape into which an inner sacrificial element in the form of a solid cylinder is inserted. Preferably, the metal preform and the inner sacrificial element have a similar axial extent, so that the cavity A is filled with the inner sacrificial element substantially over its entire length.

The person skilled in the art knows how the insertion in method step c) can be carried out.

In order to produce a shaped metal product with the smallest possible manufacturing tolerance, the combination of the two method steps d) and e) is to be emphasized in particular.

In method step d), the assembled preform is processed in such a way that the metal preform and the inner sacrificial element abut one another substantially in a form-fitting manner. If more than one inner sacrificial element is present, it is preferred that each inner sacrificial element abuts the metal preform substantially in a form-fitting manner. A substantially form-fitting abutment is achieved when there is substantially no gap or space, such as an air pocket, between the metal preform and the inner sacrificial element. Gaps between the metal preform and the inner sacrificial element result, for example, from a discrepancy in diameter between cavity A and the inner sacrificial element. In order to insert the inner sacrificial element, in method step c), smoothly and without damage, such as scratches or abrasions, into the cavity A, an outer diameter of the inner sacrificial element is preferably designed to be slightly smaller than a diameter of the cavity A. This means that the inner sacrificial element has approximately the net shape of the cavity A. Preferably, the outer diameter of the provided inner sacrificial element is smaller than the diameter of the cavity A of the metal preform by a factor in a range of 0.01% to 5%, preferably in a range of 0.01% to 3%. If the respective element has a non-circular cross section, the “diameter” is taken to be the maximum width of the element in a radial cross section.

In method step d), this discrepancy in diameters is substantially completely eliminated. This can be done, for example, by a pressing process, in particular by isostatic pressing, or a drawing process, which presses the metal preform onto the inner sacrificial element and thus substantially eliminates all gaps or spaces between the two components.

In method step e), the form-fitting preform provided in method step d) is subjected to a diffusion annealing step to obtain a composite preform. Diffusion annealing is designed in such a way that significant solid-state diffusion intentionally takes place between the metal preform and the inner sacrificial element, wherein it is irrelevant whether this is ductile diffusion, i.e. zone solid solution formation, or non-ductile diffusion, i.e. the formation of intermetallic phases. The aim of diffusion annealing is to improve the adhesion of the metal preform to the inner sacrificial element by forming a diffusion zone, preferably with a thickness in the range of 1 μm to 15 μm, between the two components, so that the original shapes of the components are retained as faithfully as possible during the subsequent method steps, in particular during the shaping in method step f), so that the smallest possible manufacturing tolerances are achieved in the final product. Without diffusion annealing, internal relative movements of the components can occur, which have a negative impact on the original shape and/or geometries of the components, in particular the metal preform, and thus result in greater manufacturing tolerances.

Diffusion annealing should not be confused with an annealing step between two drawing steps, as the latter does not result in significant solid-state diffusion due to insufficient exposure to temperature, both in terms of the temperature level and/or the time period of exposure to temperature. The exact temperature ranges and time periods at which diffusion annealing takes place depend on the materials used. A temperature range that can already cause diffusion annealing in a first material combination does not cause it in a second material combination, for example. A person skilled in the art knows under which conditions diffusion annealing takes place or does not take place for a given combination of materials. The person skilled in the art can therefore determine whether diffusion annealing or only intermediate annealing is to be carried out.

In method step f), the composite preform is reshaped into a shaped composite preform, wherein its outer diameter D1 is reduced to an outer diameter D2. For example, this can be done by means of an extrusion method. Extrusion is a method in which a block of material is pressed through a die with a desired cross section in a chamber. Pressing can be carried out using a mechanical or hydraulic press, for example.

In method step g), a composite preform disk is separated from the previously obtained shaped composite preform. Separating is used to set the desired dimensions, in particular the desired axial length of the final shaped metal products. In addition, this method step can contain further steps of shaping the obtained composite preform disk. For example, the composite preform disk, in particular the portion of the metal preform on the composite preform disk, can be milled or drilled in order to realize the desired structural features on the final metal product. For example, the material of the metal preform in the composite preform disk can be provided with a drilled hole extending axially through the composite preform disk, which represents an axially extending feedthrough in the final metal product.

In method step h), the inner sacrificial element is removed to obtain the shaped metal product with the desired dimension, geometry and/or shape. The inner sacrificial element can be removed by melting, chemical etching, or galvanic etching, for example. In one embodiment, the removal is carried out based on exploiting the different melting points of the materials used. For example, the material of the inner sacrificial element can have a lower melting point than the material of the metal preform and can be removed by selective melting. In a further preferred embodiment, the removal of the inner sacrificial element is carried out by wet chemical etching, for example by means of a strong acid such as nitric acid, sulfuric acid, hydrochloric acid or a mixture of these acids.

Preferably, the wet chemical etching is carried out using a strong, preferably aqueous, acid, such as nitric acid, sulfuric acid, hydrochloric acid or a mixture of these acids, at a temperature in a range between 18° C. and 120° C. Preferably, method step h) is carried out under the action of ultrasound, preferably in an ultrasonic bath. It can be preferable to carry out method step h) under negative pressure.

Preferably, in addition to the inner sacrificial element, the diffusion zone formed by the diffusion annealing is also removed, preferably using the same method.

For the production of a shaped metal product with a circular outer surface, the exclusive use of one or more inner sacrificial elements may be sufficient. If a shaped metal product with a non-circular outer surface is to be produced, it is preferable to additionally use an outer sacrificial element. For example, it may be preferable to use an outer sacrificial element if the metal preform has a raised part or a depression, such as a fin or a groove, on or in its outer surface. The outer sacrificial element thus serves to protect the outer surface of the metal preform during the production process, in particular if it is non-circular in shape.

An embodiment of the method is characterized in that in method step b) an outer sacrificial element having a cavity B is additionally provided, method step c) comprises producing an assembled preform by inserting the metal preform into the cavity B of the outer sacrificial element and inserting the inner sacrificial element into the cavity A of the metal preform, in method step d) the assembled preform is processed such that in the form-fitting preform the metal preform abuts the inner sacrificial element and the outer sacrificial element substantially in a form-fitting manner, and in method step h) the outer sacrificial element is additionally removed.

The method according to this embodiment comprises the following method steps:
a) providing a metal preform having a cavity A;
b) providing an outer sacrificial element having a cavity B and an inner sacrificial element;
c) producing an assembled preform by inserting the metal preform into the cavity B of the outer sacrificial element and inserting the inner sacrificial element into the cavity A of the metal preform;
d) processing the assembled preform produced in method step c) to obtain a form-fitting preform, wherein in the form-fitting preform the metal preform abuts the inner sacrificial element and the outer sacrificial element substantially in a form-fitting manner;
e) diffusion annealing of the form-fitting preform produced in method step d) to obtain a composite preform having an outer diameter D1;
f) shaping the composite preform obtained in method step e) to obtain a shaped composite preform having a smaller outer diameter D2 than the outer diameter D1 of the composite preform obtained in method step e);
g) separating a composite preform disk from the shaped composite preform obtained in method step f);
h) removing the outer sacrificial element and the inner sacrificial element from the composite preform disk obtained in method step g) to obtain the shaped metal product.

Preferably, the outer sacrificial element completely surrounds the metal preform in a radial cross-sectional view. The outer sacrificial element may have a hollow cylindrical shape. The outer shape of the outer sacrificial element preferably has a circular or oval cross section. The inner shape of the outer sacrificial element, i.e. the cavity B, can have any shape, but preferably corresponds substantially to a negative of the outer shape of the metal preform.

Preferably, the inner sacrificial element and the outer sacrificial element comprise the same material; more preferably, the inner sacrificial element and the outer sacrificial element are made of the same material.

As already mentioned, the processing in method step d) can be carried out in different ways in order to achieve a substantially form-fitting abutment of the metal preform to the inner sacrificial element and, if present, to the outer sacrificial element. For example, the processing can be carried out by a pressing process, preferably using an isostatic press.

An embodiment of the method is characterized in that the processing in method step d) comprises a drawing process, for example a wire drawing process. Preferably, the drawing process is carried out with a deformation factor between 5% and 40%, preferably between 10% and 30%. It is further preferred that the drawing process is carried out using a drawing die with an inclusion angle 2 a in a range of 5° to 20°, preferably 5° to 15°. Furthermore, it is preferred that the drawing process is carried out with a deformation factor between 5% and 40%, preferably between 10% and 30%, and using a drawing die with an inclusion angle 2 a in a range of 5° to 20°, preferably 5° to 15°.

Due to the drawing process, the form-fitting preform has a greater length and a smaller outer diameter compared to the assembled preform.

Depending on the material combination of metal preform, inner sacrificial element and, if present, outer sacrificial element, the diffusion annealing in method step e) can take place in different temperature ranges.

An embodiment of the method is characterized in that the diffusion annealing in method step e) is carried out in a temperature range of 450° C. to 1200° C., preferably in a temperature range of 450° C. to 1150° C.

Depending on the material combination of metal preform, inner sacrificial element and, if present, outer sacrificial element, the diffusion annealing in method step e) can take place over varying periods of time.

An embodiment of the method is characterized in that the diffusion annealing in method step e) is carried out over a period of time in a range from 15 min to 300 min, preferably in a range from 20 min to 260 min.

The diffusion annealing can also be divided into two or more individual steps, each of these individual steps being carried out over a period of time in a range of 15 min to 300 min, preferably in a range of 20 min to 260 min. For example, diffusion annealing takes place in two individual steps, each of which is carried out at a temperature of 800° C. over a period of 30 min.

To a certain extent, the temperature used and the duration of diffusion annealing are interrelated. For example, the duration of a diffusion annealing can be reduced if it is carried out at higher temperatures.

As already described, the shaping in method step f) can be carried out in different ways.

An embodiment of the method is characterized in that the shaping in method step f) comprises a drawing process. The drawing process may comprise a single drawing step or a plurality of individual drawing steps, i.e. at least two, preferably 3 to 50, such steps. Preferably, the drawing process is carried out with a deformation factor between 5% and 40%, preferably between 10% and 30%, per individual drawing step. Preferably, the drawing process is carried out using a drawing die with an inclusion angle of 2a in a range of 5° to 20°. Most preferably, the drawing process is carried out with a deformation factor between 5% and 40%, preferably between 10% and 30%, per individual drawing step, and using a drawing die with an inclusion angle 2a in a range of 5° to 20°. The person skilled in the art knows how such a drawing process can be carried out.

An embodiment of the method is characterized in that the composite preform is annealed in method step f) between at least one, preferably between all individual drawing steps if more than one individual drawing step is carried out. This annealing is used to remove as much material stress as possible that may be present in the composite preform after a drawing step. The annealing must be selected in such a way that substantially no solid-state diffusion occurs between the metal preform and the inner sacrificial element and, if present, also the outer sacrificial element. The exact conditions of annealing depend on the materials used, taking into account the degree of deformation of the drawing step, and are known to the person skilled in the art.

The shaped metal products produced by the method according to the invention are preferably suitable for use in a medical product, in particular in an active implantable medical product. Preferred active medical products include, for example, pacemakers, cardiac defibrillators, neurostimulators, cochlear implants, implantable cardioverters, nerve, brain, organ or muscle stimulators, as well as implantable monitoring devices, hearing aids, retinal implants, muscle stimulators, implantable drug pumps, artificial hearts, bone growth stimulators, prostate implants, gastric implants, or the like. Preferably, the active medical product is a pacemaker, an electroporation system (pulse field ablation), or a neuromodulator.

Preferably, the shaped metal product is used as an electrode, preferably as a ring electrode or as a segmented electrode, in one of the medical products described above.

A further subject matter of the invention relates to a method for producing a composite preform according to the invention, comprising
a) a metal preform having a cavity A′,
b) an inner sacrificial element arranged in the cavity A′ of the metal preform,
characterized in that an inner diffusion zone having a thickness in a range of 1 μm to 15 μm is formed at an interface between the metal preform and the inner sacrificial element.

The composite preform is preferably produced by means of an embodiment of the method described above.

The composite preform has an inner diffusion zone with a thickness in a range of 1 μm to 15 μm, preferably in a range of 1 μm to 10 μm, most preferably in a range of 2 μm to 9 μm, at an interface between the metal preform and the inner sacrificial element, preferably at the entire interface between the metal preform and the inner sacrificial element. The inner diffusion zone comprises both the elements of the metal preform and of the inner sacrificial element, preferably in the form of a ductile diffusion zone, i.e. comprising solid solutions of the elements, or a non-ductile diffusion zone, i.e. comprising intermetallic phases of the elements. The inner diffusion zone ensures improved adhesion of the metal preform and the inner sacrificial element, so that the original shapes of the components remain as unchanged as possible during the subsequent method steps for producing the shaped metal product and a shaped metal product can be produced with the lowest possible manufacturing tolerance.

The described thickness range of the inner diffusion zone has proven to be advantageous, as it represents a good compromise between sufficient adhesion of the metal preform to the inner sacrificial element and, at the same time, the smallest possible thickness of the diffusion zone. Thinner diffusion zones are easier to remove and ensure lower manufacturing tolerances during removal. Preferably, the final shaped metal product has substantially no inner diffusion zone.

If the composite preform has more than one inner sacrificial element, it is preferred for an inner diffusion zone to be formed between all inner sacrificial elements and the metal preform.

The person skilled in the art knows how to determine a diffusion zone and its thickness. For example, the diffusion zone and its thicknesses in a cross section of the composite preform can be determined using energy dispersive X-ray spectroscopy (EDX).

An embodiment of the composite preform is characterized in that the inner diffusion zone comprises an intermetallic phase.

An embodiment of the composite preform is characterized in that the composite preform comprises an outer sacrificial element having a cavity B′, wherein the metal preform is arranged in the cavity B′ of the outer sacrificial element and an outer diffusion zone with a thickness in a range of 1 μm to 15 μm, preferably in a range of 1 μm to 10 μm, most preferably in a range of 2 μm to 9 μm, is formed at an interface of the metal preform and the outer sacrificial element.

The outer diffusion zone comprises both the elements of the metal preform and of the outer sacrificial element, preferably in the form of a ductile diffusion zone, i.e. comprising solid solutions of the elements, or a non-ductile diffusion zone, i.e. comprising intermetallic phases of the elements. The outer diffusion zone ensures improved adhesion of the metal preform and the outer sacrificial element, so that the original shapes of the components remain as unchanged as possible during the subsequent method steps for producing the shaped metal product and a shaped metal product can be produced with the lowest possible manufacturing tolerance.

The described thickness range of the outer diffusion zone has proven to be advantageous, as it represents a good compromise between sufficient adhesion of the metal preform to the outer sacrificial element and, at the same time, the smallest possible thickness of the diffusion zone. Thinner diffusion zones are easier to remove and ensure lower manufacturing tolerances during removal. Preferably, the final shaped metal product has substantially no outer diffusion zone.

If the composite preform has more than one outer sacrificial element, it is preferred that an outer diffusion zone is formed between all outer sacrificial elements and the metal preform.

An embodiment of the composite preform is characterized in that the outer diffusion zone comprises an intermetallic phase.

A further subject matter of the invention relates to a use of the composite preform described above for producing a medical product, preferably for producing a medical ring electrode or a segmented medical electrode.

### EXAMPLE

An exemplary shaped metal product was produced using the following method steps and under the following conditions.

A cylindrical outer sacrificial element with a length of 230 mm, an outer diameter of 14.5 mm and an inner square cavity B with an edge length of 7.91 mm was produced from a block of nickel by means of electrical discharge machining (EDM). Furthermore, two inner sacrificial elements, namely a rectangular one (3.78×3.78 mm) and an isosceles triangular one (5.87×4.15 mm), were produced from the same material by means of electrical discharge machining. Both had a length of 230 mm.

A rectangular metal preform having a square cavity A1 as well as an isosceles triangular cavity A2 was made of a platinum-iridium alloy with a percentage by weight of 10% iridium (PtIr10) and was also produced by means of electrical discharge machining. The outer edge length of the metal preform was 7.91×7.91 mm, the outer edge length of the cavity A1 was 3.81 mm, and the triangular cavity A2 had an edge length of 5.9×4.18 mm. The length of the metal preform was 230 mm.

The two inner sacrificial elements were placed in the cavities A1 and A2 of the metal preform and the latter was placed in the cavity B of the outer sacrificial element to obtain an assembled preform.

The assembled preform was subjected to a pressing process by means of a drawing process, which substantially removed the gap between the components. The pressing process transformed the assembled preform into a form-fitting preform.

The form-fitting preform was then diffusion-annealed at a temperature of 600° C. for a duration of 120 min to obtain a composite preform. A diffusion zone with a thickness of approximately 5 μm was formed between the metal preform and the adjacent sacrificial element.

The composite preform was subjected to a drawing process. In a first drawing step, the diameter of the composite preform of 13.1 mm was drawn in multiple drawing steps until a final diameter of the resulting formed composite preform of 1.74 mm was obtained. Each time a total deformation degree of 60% was achieved, the composite was subjected to an intermediate annealing of 30 minutes at 500° C. in order to bring the material composite back into a ductile state for the further forming steps.

A shaped metal product was obtained by separating a composite preform disk from the shaped composite preform and removing the sacrificial elements by etching, as described in US2019255317A1, which is hereby incorporated by reference in its entirety.

As a comparative example, another shaped metal product was produced according to the method described above and with the same dimensions, but omitting the diffusion annealing step. In the following table, dimensions of the example and the comparative example were

compared and a shrinkage factor was calculated from the dimensions of the starting components and the measured dimensions of the shaped composite preform (once with and once without diffusion annealing). This shrinkage factor calculated by measurements was compared with a theoretical shrinkage factor resulting from the initial and final dimensions of the outer sacrificial element.

TABLE 1
Dimensions of the comparative example and
of the exemplary shaped metal product.

| | Comparative | | | |
|---|---|---|---|---|
| | example | | Example | |
| | (shaped | | (shaped | |
| | composite | | composite | |
| | preform | Shrinkage | preform | |
| | without | factor of | with | Shrinkage |
| | diffusion | comparative | diffusion | factor of |
| | annealing) | example | annealing) | example |
| Outer diameter of | 1.740 mm | 8.33/1 | 1.740 mm | 8.33/1 |
| outer sacrificial | | | | |
| element | | | | |
| Outer edge of metal | 0.952 mm | 8.31/1 | 0.951 mm | 8.32/1 |
| preform | | | | |
| Edge length 1 of | 0.458 mm | 8.25/1 | 0.454 mm | 8.32/1 |
| rectangular inner | | | | |
| sacrificial element | | | | |
| Edge length 2 of | 0.458 mm | 8.25/1 | 0.454 mm | 8.32/1 |
| rectangular inner | | | | |
| sacrificial element | | | | |
| Edge length 1 of | 0.710 mm | 8.26/1 | 0.705 mm | 8.33/1 |
| isosceles triangular | | | | |
| inner sacrificial | | | | |
| element | | | | |
| Edge length 2 of | 0.504 mm | 8.23/1 | 0.499 mm | 8.32/1 |
| isosceles triangular | | | | |
| inner sacrificial | | | | |
| element | | | | |
| Edge length 3 of | 0.504 mm | 8.23/1 | 0.499 mm | 8.32/1 |
| isosceles triangular | | | | |
| inner sacrificial | | | | |
| element | | | | |
| Smallest shrinkage | | 8.23/1 | | 8.32/1 |
| factor | | | | |
| Largest shrinkage | | 8.33/1 | | 8.33/1 |
| factor | | | | |
| Average shrinkage | | 8.27/1 | | 8.32/1 |
| factor | | | | |

Table 1 shows the improved manufacturing tolerance of the method with diffusion annealing compared to the same method without diffusion annealing. The theoretical shrinkage factor is 8.33, which corresponds to the ratio of the starting diameter to the final diameter of the outer sacrificial element (see first row with measured values).

The process stability specified here is calculated from the ratio of the minimum to maximum shrinkage factor actually achieved. The test design without diffusion annealing results in a process stability of 98.7%. In comparison, the test design using diffusion annealing described here results in a process stability of 99.9%.

### BRIEF DESCRIPTION

The invention is further illustrated by way of example below by means of figures. The invention is not limited to the figures.

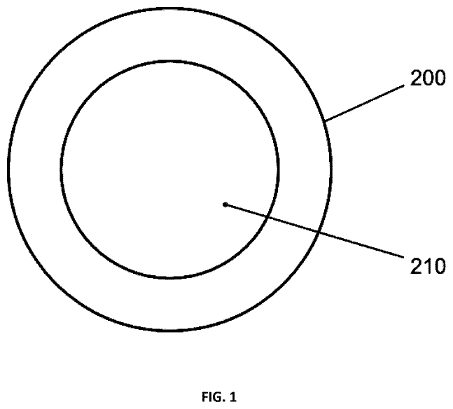

FIGS. 1 to 6 illustrate an exemplary method for producing a shaped metal product.

In the drawings:

FIG. 1 shows an exemplary metal preform having a cavity A for producing a shaped metal product,

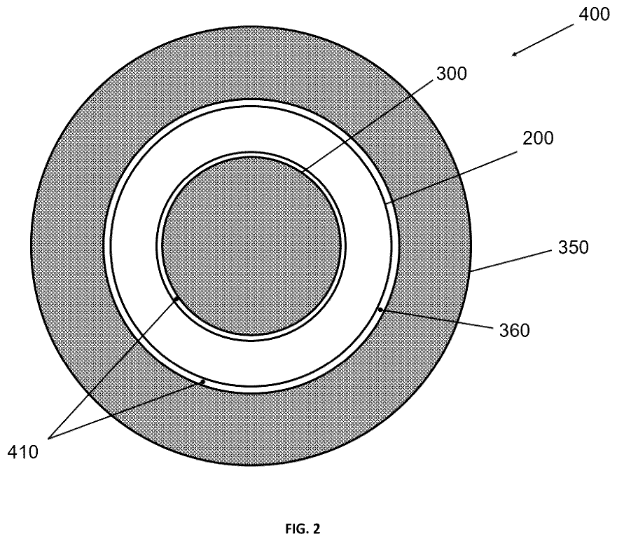

FIG. 2 shows an assembled preform comprising the metal preform of FIG. 1 and an inner sacrificial element and an outer sacrificial element,

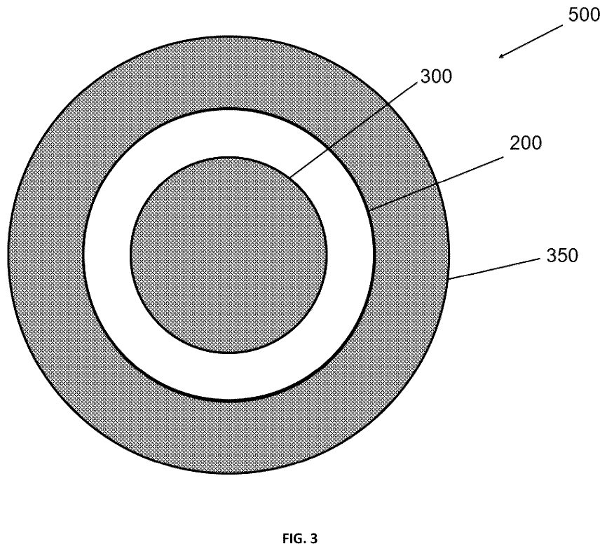

FIG. 3 shows a form-fitting sacrificial element obtained by processing the assembled preform from FIG. 2,

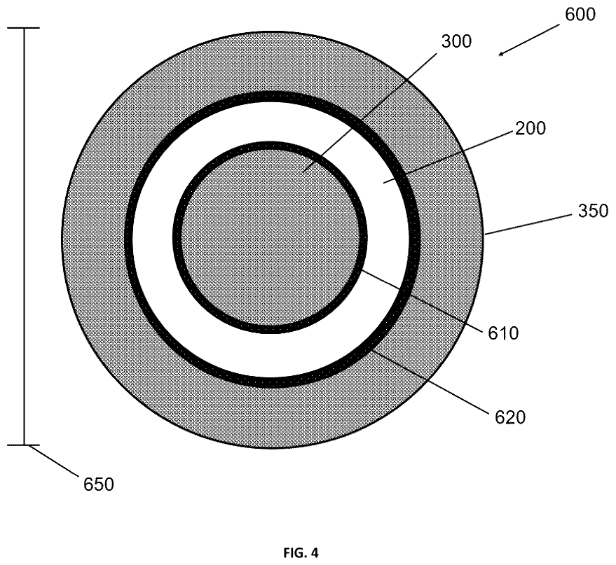

FIG. 4 shows a composite preform obtained by means of diffusion annealing of the assembled preform of FIG. 3,

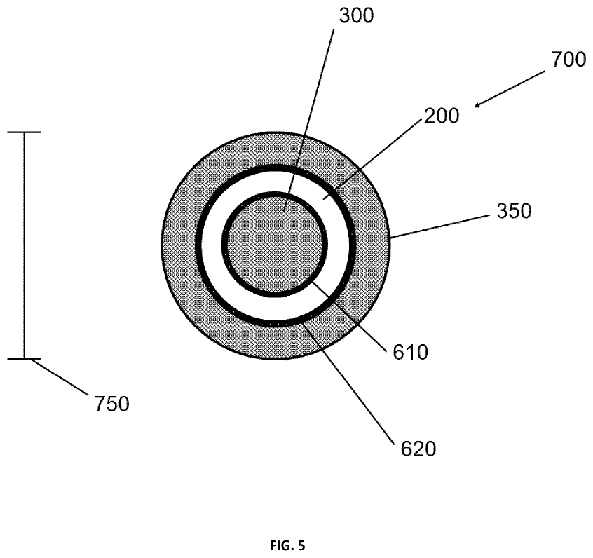

FIG. 5 shows a shaped composite preform obtained by shaping the composite preform of FIG. 4,

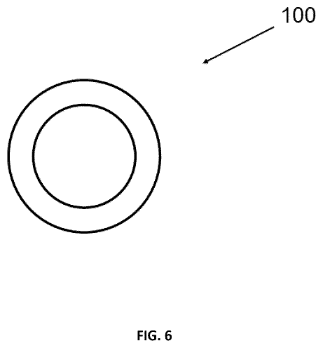

FIG. 6 shows the shaped metal product, and

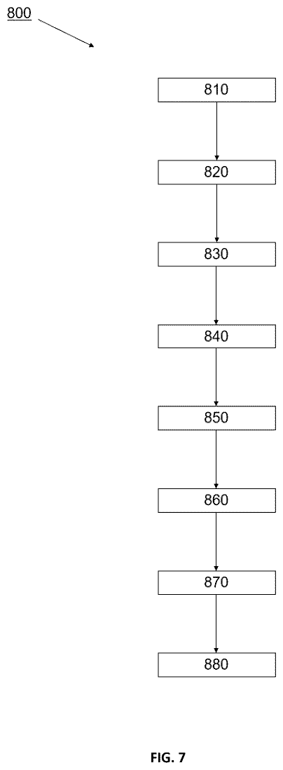

FIG. 7 is an exemplary flow chart of a method for producing a shaped metal product.

### DETAILED DESCRIPTION

FIG. 1 shows an exemplary metal preform 200 for producing a shaped metal product in a front view along a longitudinal axis of the metal preform 200. In the embodiment shown, the metal preform 200 has a hollow cylindrical configuration which encloses a cylindrical cavity A 210.

In the embodiment shown, the metal preform 200 is made of a platinum-iridium alloy with a percentage by weight of 90 weight percent platinum and 10 weight percent iridium (PtIr10).

FIG. 2 shows an assembled preform 400 as an intermediate product of the method for producing the shaped metal product, in a front view. The assembled preform 400 comprises the metal preform 200 of FIG. 1, wherein a cylindrical inner sacrificial element 300 is arranged within the cavity A 210 (see FIG. 1) of the metal preform 200. The assembled preform 400 also has a hollow cylindrical outer sacrificial element 350, wherein the metal preform 200 and the inner sacrificial element 300 are arranged within a cavity B 360 of the outer sacrificial element. Between the metal preform 200 and the inner sacrificial element 300, as well as between the metal preform 200 and the outer sacrificial element 350, there is a gap 410 which results from a diameter discrepancy of the components 200, 300, 350 of the assembled preform 400. The diameter discrepancy ensures a simplified and non-destructive assembly of the components 200, 300, 350 to form the assembled preform 400.

In the embodiment shown, the inner sacrificial element 300 and the outer sacrificial element 350 are made of nickel.

FIG. 3 shows a form-fitting preform 500 obtained by processing the assembled preform 400 from FIG. 2. In contrast to the assembled preform 400 of FIG. 2, the individual components 200, 300, 350 are abutting one another in a form-fitting manner, i.e. the inner sacrificial element 300 abuts the metal preform 200 in a form-fitting manner, and the metal preform 200 abuts the outer sacrificial element 350 in a form-fitting manner. A form-fitting abutment exists if there is substantially no gap between the components 200, 300, 350 (see FIG. 2). The processing substantially does not result in a materially bonded connection between the components 200, 300, 350.

In the embodiment shown, the form-fitting preform 500 was obtained by isostatic pressing of the assembled preform 400.

FIG. 4 shows a composite preform 600 obtained by diffusion annealing of the form-fitting preform 500 of FIG. 3. As a result of the diffusion annealing, an inner diffusion zone 610 has formed between the metal preform 200 and the inner sacrificial element 300 and an outer diffusion zone 620 has formed between the metal preform 200 and the outer sacrificial element 350. In the embodiment shown, diffusion annealing took place at a temperature of 650° C. over a period of 120 min, resulting in the formation of a diffusion zone 610, 620 with a thickness of 5 μm. Due to the material combination of metal preform 200 and the two sacrificial elements 300, 350, the diffusion zones 610, 620 each comprise solid solutions.

The composite preform 600 has an outer diameter D1 650.

FIG. 5 shows a shaped composite preform 700 produced by shaping the composite preform 600 of FIG. 4. As a result of the shaping, the shaped composite preform 700 has a reduced outer diameter D2 750 compared to the outer diameter D1 650 (see FIG. 4). Compared to the composite preform 600, the shaped composite preform 700 is “stretched” along the longitudinal axis, i.e. it has a greater axial extent than the composite preform 600. In the embodiment shown, the shaping was carried out by a drawing process.

FIG. 6 shows the produced shaped metal product 100. The shaped metal product 100 was produced from the shaped composite preform 700 shown in FIG. 5 by separating a composite preform disk with the desired axial extent from the shaped composite preform 700 and then removing the inner sacrificial element 300, the outer sacrificial element 350, the inner diffusion zone 610 and the outer diffusion zone 620 (see FIGS. 4 and 5). In the embodiment shown, these elements were etched away using wet chemical methods.

FIG. 7 shows a flow chart of an exemplary method 800 for producing a shaped metal product 100, comprising the method steps 810 to 880.

In a method step 810, a metal preform 200 having a cavity A 210 is provided. The metal preform 200 may have only one or more than one cavity A 210. If there are several cavities A 210, they can be isolated from each other or connected to each other.

In a method step 820, at least one inner sacrificial element 300 is provided. The inner sacrificial element 300 preferably comprises a different material than the metal preform 200. In one embodiment of method step 820, the latter also comprises providing at least one outer sacrificial element 350 having a cavity B 360. The outer sacrificial element 350 preferably comprises a different material than the metal preform 200, and preferably the same material as the inner sacrificial element 300.

In a method step 830, an assembled preform 400 is produced by inserting or pushing the inner sacrificial element 300 into the cavity A 210 of the metal preform 200. If an outer sacrificial element 350 is used, the metal preform 200 and the inner sacrificial element 300 are also arranged in the cavity B 360 of the outer sacrificial element 350. To facilitate the production of the assembled preform 400, the diameters of the components 200, 300 and, if present, 350, are selected such that there is a gap 410 between them.

In a method step 840, the assembled preform 400 is processed to obtain a form-fitting preform 500. In the form-fitting preform, the gap 410 between the metal preform 200 and the inner sacrificial element 300 and, if present, between the metal preform 200 and the outer sacrificial element 350, is substantially eliminated by the processing. Preferably, the processing is a pressing operation from the outside onto the assembled preform 400 and/or a drawing process on the assembled preform 400. The processing serves in particular to prepare for method step 850.

In a method step 850, the form-fitting preform 500 is diffusion-annealed to obtain a composite preform 600. The composite preform has an inner diffusion zone 610 between the metal preform 200 and the inner sacrificial element 300. If an outer sacrificial element 350 is used, the composite preform 600 has an outer diffusion zone 620 between the metal preform and the outer sacrificial element 350. The inner diffusion zone 610 and, if present, the outer diffusion zone 620, improve the adhesion of the components 200, 300, and, if present, 350, so that their geometry is substantially not distorted by the subsequent method steps and a shaped metal product 100 can be produced with the smallest possible manufacturing tolerances.

In a method step 860, the composite preform 600 is shaped to form a shaped composite preform 700. Here an outer diameter D1 650 of the composite preform 600 is reduced to an outer diameter D2 750 of the shaped composite preform 700. The shaping preferably comprises a drawing process.

In a method step 870, a composite preform disk is separated from the shaped composite preform 700 having a desired thickness of the shaped metal product 100. The separating preferably comprises cutting, for example by means of a laser. Preferably, a plurality, for example 50 to 10,000, of composite preform disks can be separated from a single composite preform 700. This simplifies the production of the shaped metal product 100 and thus reduces the production costs.

In a method step 880, the inner sacrificial element 300 and, if present, the outer sacrificial element 350, preferably together with all diffusion zones 610, 620, are removed from the composite preform disk to obtain the shaped metal product 100. Preferably, the removal comprises an etching process using an acid or a base.

REFERENCE SIGNS

| | |
|---|---|
| 100 | shaped metal product |
| 200 | metal preform |
| 210 | cavity A |
| 300 | inner sacrificial element |
| 350 | outer sacrificial element |
| 360 | cavity B |
| 400 | assembled preform |
| 410 | gap |
| 500 | form-fitting preform |
| 600 | composite preform |
| 610 | inner diffusion zone |
| 620 | outer diffusion zone |
| 650 | outer diameter D1 |
| 700 | shaped composite preform |
| 750 | outer diameter D2 |
| 800 | method |
| 810 | providing the metal preform |
| 820 | providing the inner sacrificial element |
| 830 | producing the assembled preform |
| 840 | processing the assembled preform |
| 850 | diffusion annealing |
| 860 | shaping |
| 870 | separating |
| 880 | removing |

1. A method for producing a shaped metal product, preferably for a medical device, having the following method steps:
a) providing a metal preform having a cavity A;
b) providing an inner sacrificial element;
c) producing an assembled preform by inserting the inner sacrificial element into the cavity A of the metal preform;
d) processing the assembled preform produced in method step c) to obtain a form-fitting preform, wherein in the form-fitting preform the metal preform abuts the inner sacrificial element substantially in a form-fitting manner;
e) diffusion annealing of the form-fitting preform produced in method step d) to obtain a composite preform having an outer diameter D1;
f) shaping the composite preform obtained in method step e) to obtain a shaped composite preform having a smaller outer diameter D2 than the outer diameter D1 of the composite preform obtained in method step e);
g) separating a composite preform disk from the shaped composite preform obtained in method step f);
h) removing the inner sacrificial element from the composite preform disk obtained in method step g) to obtain the shaped metal product.

2. The method according to claim 1, wherein in method step b) an outer sacrificial element having a cavity B is additionally provided, method step c) comprises producing an assembled preform by inserting the metal preform into the cavity B of the outer sacrificial element and inserting the inner sacrificial element into the cavity A of the metal preform, in method step d) the assembled preform is processed such that in the form-fitting preform the metal preform abuts the inner sacrificial element and the outer sacrificial element substantially in a form-fitting manner, and in method step h) the outer sacrificial element is additionally removed.

3. The method according to claim 1, wherein the processing in method step d) comprises a drawing process, wherein the drawing process is preferably carried out with a deformation factor between 5% and 40% and/or using a drawing die with an inclusion angle 2 a in a range of 5° to 20° or wherein the processing in method step d) comprises a pressing process, in particular isostatic pressing.

4. The method according to claim 1, wherein the diffusion annealing in method step e) is carried out in a temperature range of 450° C. to 1200° C., preferably in a temperature range of 450° C. to 1150° C.

5. The method according to claim 1, wherein the diffusion annealing in method step e) is carried out over a period of time in a range from 15 min to 300 min, preferably in a range from 20 min to 260 min.

6. The method according to claim 1, wherein the shaping in method step f) comprises a drawing process, wherein the drawing process is preferably carried out with a deformation factor between 5% and 40%, preferably between 10% and 30%, per individual drawing step and/or using a drawing die with an inclusion angle 2 a in a range of 5° to 20°.

7. The method according to claim 6, wherein the drawing process in method step f) comprises 3 to 50 individual drawing steps.

8. The method according to claim 6, wherein the composite preform is annealed in method step f) between the individual drawing steps of the drawing process.

9. The method according to claim 1, wherein the metal preform comprises a metal selected from the group consisting of platinum, gold, iridium, steel, titanium, hafnium, niobium, cobalt, nickel, chromium, zirconium, rhenium, tungsten, molybdenum, and alloys of at least one of these metals, preferably selected from the group consisting of nickel-cobalt alloys, steel, platinum, and platinum-iridium alloys.

10. The method according to claim 1, wherein the composite preform is shaped in the method such that it assumes a smaller dimension without substantially altering its overall geometry.

11. A composite preform for producing a shaped metal product, comprising
a) a metal preform having a cavity A′,
b) an inner sacrificial element arranged in the cavity A′ of the metal preform,
wherein an inner diffusion zone with a thickness in a range of 1 μm to 15 μm is formed at an interface between the metal preform and the inner sacrificial element.

12. The composite preform according to claim 11, wherein the inner diffusion zone comprises an intermetallic phase.

13. The composite preform according to claim 11, comprising an outer sacrificial element having a cavity B′, wherein the metal preform is arranged in the cavity B′ of the outer sacrificial element and an outer diffusion zone with a thickness in a range of 1 μm to 8 μm is formed at an interface of the metal preform and the outer sacrificial element.

14. The composite preform according to claim 13, wherein the outer diffusion zone comprises an intermetallic phase.

15. A use of a composite preform according to claim 11 for producing a medical product, preferably for producing a medical ring electrode or a segmented medical electrode.
